(19) United States
(12) Patent Application Publication
(10) Pub. No.: US 2019/0214110 A1
(43) Pub. Date: Jul. 11, 2019

(54) DETECTION OF INSUFFICIENT HOMOLOGY REGIONS IN A REFERENCE SEQUENCE

(71) Applicant: Seven Bridges Genomics Inc., Cambridge, MA (US)

(72) Inventor: Yilong Li, Cambridge, MA (US)

(73) Assignee: Seven Bridges Genomics Inc., Cambridge, MA (US)

(21) Appl. No.: 16/213,351

(22) Filed: Dec. 7, 2018

Related U.S. Application Data
(60) Provisional application No. 62/595,603, filed on Dec. 7, 2017.

(51) Int. Cl. G16B 30/10; C12Q 1/6809; G16B 20/20; G16B 20/10; G16B 25/10; G16B 40/10; G16B 45/00; G16B 50/20; G16B 50/30; G16B 35/20

(52) U.S. Cl. CPC G16B 30/10; C12Q 1/6809; G16B 20/20; G16B 20/10; G16B 35/20; G16B 40/10; G16B 45/00; G16B 50/20; G16B 50/30; G16B 25/10

(57) ABSTRACT

Method and apparatus for detecting insufficient homology regions of a reference sequence are described herein. The method can identify an insufficient homology region within a reference sequence with respect to a genomic sample of a subject by determining the extent of abnormality of a particular combination of features exhibited by an aligned sequence read. The method can include obtaining an initial alignment based on a reference sequence and sequence reads derived from a genomic sample of a subject, determining an expected background level of alignment abnormalities in the initial alignment, determining an unexpectedness of observed alignment abnormalities for each sequence read, identifying insufficient homology regions within the reference sequence, and tagging the identified insufficient homology region for a remedial action.

### RELATED APPLICATIONS

This application claims priority to U.S. Provisional Patent Application No. 62/595,603, filed Dec. 7, 2017, the contents of which are hereby incorporated by reference in their entirety.

### FIELD

The invention relates to methods, systems, and apparatuses relating to DNA sequencing. Specifically, the invention relates to evaluating reference sequences for use in genome sequencing.

### BACKGROUND

Next Generation Sequencing (NGS) (e.g., whole-genome shotgun sequencing, pyrosequencing, ion semiconductor sequencing, sequencing by synthesis) is a powerful tool for sequencing an entire genome by generating a large number of sequence reads spanning a whole genome and assembling the sequence reads into longer contigs.

One approach to genome sequencing using NGS is “de novo sequencing,” where sequence reads are assembled into longer contigs by lining up overlapping ends of the sequence reads. However such an approach requires much computing power and time, for example, due to the high number of contigs and scaffolds that are generated. In contrast reference-assisted assembly provides a more efficient way to assemble genome-wide or chromosome-wide sequencing by arranging contigs and scaffolds into putative chromosomes using information from a reference sequence. Thus, the current paradigm for whole-genome sequencing analysis using NGS is the reference-assisted assembly. In order for reference-assisted assembly to work well, the genome being sequenced must be sufficiently similar to the reference genome.

However, while an individual's genome may have high homology in the vast majority of the reference genome, often there are regions in which homology is insufficient. For example, persons with African ancestry have many regions of the genome that widely diverge from commonly used reference sequences, such as the GRCh38 human genome reference. In these insufficient homology regions (“IHRs”) of the reference genome, a sequence alignment program (or “aligner”) may cause sequence reads to align incorrectly, resulting in erroneous variant calls. In such instances, some aligners will attempt to perform a de novo reassembly in these regions based on various measures. For example, the Genome Analysis Toolkit (GATK) Haplotype Caller calculates a per-base “activity” measure based on the variability of individual bases aligned at each position. More specifically, GATK HaplotypeCaller may be used to identify IHRs (“Active Regions”) in which the samples being analyzed show substantial evidence of variation relative to the reference sequence in the following way: (1) based on a reference alignment, GATK HaplotypeCaller computes an activity score (between 0 and 1) for each individual genome position; (2) activity scores are then “smoothed” (i.e., averaged over a sliding window) to yield an “activity profile.” Regions exceeding a threshold value are selected for local reassembly; (3) once regions are selected, GATK HaplotypeCaller would proceed by performing local reassembly and then determining a list of possible haplotypes. The activity score represents the probability that the position contains a variant. The raw activity profile, which is generated from the activity scores, is a function of activity per position, which is an estimate of the variability at that position represented by the aligned bases at that position (A, C, G, or T). The raw activity profile is smoothed to produce the activity profile, from which Active Region thresholds and intervals are determined.

However, GATK's HaplotypeCaller produces a simple measure that often misses other regions of insufficient homology. In other words, when a user uses GATK HaplotypeCaller to detect IHRs of a reference genome sequence, the user may not detect some such regions. When used in reference-assisted assembly, a reference genome sequence that has undetected IHRs with respect to a genomic sample set of reads may result in false-positive and/or incorrect variant calls. In addition, presently available aligners such as GATK HaplotypeCaller may identify regions of a reference sequence as IHRs, for example due to sequencing errors, when these regions have sufficient homology to genomic samples. Accordingly, there is a need for improvements in identifying regions of low homology in reference-assisted assemblies. Therefore, an improved method for identifying IHRs in a reference genome sequence is needed to provide an improved reference-assisted assembly of a genomic sample of a subject with fewer false-positives and a more reliable genetic sequence analysis of a genomic sample of a subject.

### SUMMARY

The invention is based, at least in part, upon the discovery that IHRs of a reference sequence with respect to a genomic sample of a subject can be more successfully identified by analyzing the alignment abnormality features of corresponding sequence reads. Using this approach, a user can identify IHRs that would otherwise not have been identified using presently available alternatives such as GATK HaplotypeCaller. Additionally, using this approach, a user can prevent identifying non-IHRs as IHRs as it is the case with GATK HaplotypeCaller.

In various aspects, the invention provides a computer-implemented method for identifying an insufficient homology region (“IHR”) within a reference sequence with respect to a genomic sample of a subject, the method being based on determining the extent of abnormality of a particular combination of features exhibited by an aligned sequence read, the method comprising: (a) obtaining an initial alignment based on a reference sequence, the initial alignment comprising a plurality of sequence reads derived from the genomic sample of the subject and the reference sequence; (b) determining an expected background level for each of two or more types of alignment abnormalities in the initial alignment, the background levels being specific to the genomic sample; (c) determining an unexpectedness of observed alignment abnormalities for each sequence read by calculating a score based on the two or more types of observed alignment abnormalities in each sequence read; (d) identifying the insufficient homology region within the reference sequence, wherein the insufficient homology region is associated with sequence reads having a lower combined score than a threshold score; and (e) tagging the identified insufficient homology region for a remedial action.

In various aspects, the invention provides a data processing system for designating an insufficient homology region of a reference sequence, the system comprising: at least one memory operable to store bioinformatics data; and a processor communicatively coupled to the at least one memory, the processor being configured to: (a) obtain an initial alignment based on a reference sequence, the initial alignment comprising a plurality of sequence reads derived from the genomic sample of the subject and the reference sequence; (b) determine an expected background level for each of two or more types of alignment abnormalities in the initial alignment, the background levels being specific to the genomic sample; (c) determine an unexpectedness of observed alignment abnormalities for each sequence read by calculating a score based on the two or more types of observed alignment abnormalities in each sequence read; (d) identify the insufficient homology region within the reference sequence, wherein the insufficient homology region is associated with sequence reads having a lower combined score than a threshold score; and (e) tag the identified insufficient homology region for a remedial action.

In various aspects, the invention provides a non-transitory computer-readable medium comprising instructions which, when implemented by one or more computers, cause the one or more computers to: (a) obtain an initial alignment based on a reference sequence, the initial alignment comprising a plurality of sequence reads derived from the genomic sample of the subject and the reference sequence; (b) determine an expected background level for each of two or more types of alignment abnormalities in the initial alignment, the background levels being specific to the genomic sample; (c) determine an unexpectedness of observed alignment abnormalities for each sequence read by calculating a score based on the two or more types of observed alignment abnormalities in each sequence read; (d) identify the insufficient homology region within the reference sequence, wherein the insufficient homology region is associated with sequence reads having a lower combined score than a threshold score; and (e) tag the identified insufficient homology region for a remedial action.

It should be understood by those skilled in the art, any of the aspects above can be combined with any one or more of the features below.

In various embodiments, the initial alignment comprises a reference-assisted assembly of the sequence reads to the reference sequence.

In various embodiments, the types of observed alignment abnormalities comprise at least two of: (a) abnormal read pairing type alignment abnormality; (b) soft clipping type alignment abnormality; (c) mismatch type alignment abnormality; (d) insertion type alignment abnormality; and (e) deletion type alignment abnormality.

In various embodiments, if the alignment abnormality is a mismatch type abnormality, then the step of determining the expected background levels comprises assessing the expected background level of the alignment abnormality based on the following covariates: (a) base position of the abnormality with respect to the sequence read; and (b) base quality of the base position of the abnormality.

In various embodiments, if the alignment abnormality is an insertion type abnormality or a deletion type abnormality, then the step of determining the expected background levels comprises assessing the expected background level of an alignment abnormality based on a base position of the abnormality with respect to the sequence read.

In various embodiments, if the alignment abnormality is an abnormal read pairing type abnormality, then the step of determining the expected background levels comprises assessing the expected background levels of the alignment abnormality by dividing a total count of the abnormality by a total number of the sequence reads.

In various embodiments, if the alignment abnormality is a soft-clipping type of abnormality, then the step of determining the expected background levels comprises assessing the expected background level of the alignment abnormality based on the following covariates: (a) length of a soft clip of a read; and (b) determination of whether the abnormality occurs at the 3′ end of the read or the 5′ end of the read.

In various embodiments, calculating a score based on the two or more types of observed alignment abnormalities in each sequence read comprises: (a) calculating an individual p-value for each of two or more types of observed alignment abnormalities in each sequence read; and (b) combining a plurality of individual p-values to generate a single combined p-value for the observed alignment abnormalities for each sequence read; wherein the insufficient homology region has a lower combined p-value than a threshold p-value.

In various embodiments, the p-value of each type of observed alignment abnormality in each sequence read is calculated by performing one of: (a) p=Pr(X≥o), wherein X˜Poisson(Σ_{i∈A}λᵢ), A={i: −10 log₁₀(λᵢ)≥20}, and o=Σ_{i∈A}I(mismatch at position i), λᵢ is the base quality at position i and I(⋅) is the indicator function; (b) p=Σ_{i∈A}rᵢ, wherein A={i:rᵢ≤rₓ}, wherein p is calculated separately for first and second reads and 5′ and 3′ ends of the sequence reads, and rᵢ is the rate at which the order and end of the reads are clipped for i bases, which may be estimated from the alignment data itself; (c) p=1 if the sequence read is free of an abnormality, and p=r_{abnormal read pairing} if the sequence read is abnormally paired in a read pair, where r_{abnormal read pairing} is the overall rate of a mapped read pair being abnormally paired; and (d) p=Pr(X≥o), wherein o is the number of insertions or deletions (“indels”) observed in a read, X˜Poisson(Σᵢλᵢ) and λᵢ are the respective background likelihoods for having an indel aligned (i.e., non-soft-clipped) at position i, and wherein background rates for indels are computed separately for each of the two strands of the read.

In various embodiments, the step of combining a plurality of individual p-values to generate a single combined p-value for the observed alignment abnormalities for each sequence read is performed using Fisher's method.

In various embodiments, the step of combining a plurality of individual p-values comprises a plurality of steps such that at each step, some individual p-values are combined first and the resultant intermediate combined p-values are combined with some or all of remaining individual p-values.

In various embodiments, the step of generating the combined p-value further comprises the step of generating the combined p-value further comprises smoothing the combined p-value for the observed alignment abnormalities of each sequence window.

In various embodiments, the remedial action comprises: (a) a de novo local assembly of the identified insufficient homology region; (b) a masking of sequences of the insufficient homology region; or (c) a determination by a user on the appropriateness of the reference sequence for use with respect to the genomic sample of the subject.

These and other advantages of the present technology will be apparent when reference is made to the accompanying drawings and the following description.

### BRIEF DESCRIPTION OF THE DRAWINGS

FIG. 1 is a schematic diagram of a data processing system for designating an insufficient homology region of a reference sequence.

FIG. 2 is a flow chart illustrating an embodiment of a method for identifying insufficient homology regions using the computer system of FIG. 1.

FIG. 3 is a schematic diagram illustrating a per-read approach of determining the likelihood of observing different types of alignment abnormality features of sequence reads in an initial alignment, according to invention principles.

FIGS. 4-7 are examples comparing IHRs that are identified using an algorithm according to the present invention with the IHRs that are identified using GATK HaplotypeCaller.

While the invention comprises embodiments in many different forms, they are shown in the drawings and will herein be described in detail several specific embodiments with the understanding that the present disclosure is to be considered as an exemplification of the principles of the technology and is not intended to limit the invention to the embodiments illustrated.

### DETAILED DESCRIPTION

The invention is based, at least in part, upon the discovery that IHRs of a reference sequence can be identified more successfully than currently available methods such as those used in GATK HaplotypeCaller by analyzing the alignment abnormality features of sequence reads on a per-read basis, rather than a per-position basis. Further, the invention allows flagging the identified IHRs of the reference genome for a remedial action such as masking such regions from variant calling, reassembling the sequence reads aligned to the IHR de novo, performing targeted resequencing, and the like.

The invention provides a novel approach to detecting IHRs by considering various properties of individual sequence reads in the reference alignment, and estimating the likelihood that an alignment pattern of a particular sequence read is observed given an estimated background rate for different alignment anomalies. These results can be summarized in, e.g., sliding windows across the reference alignment, identifying IHRs as those in which the values within a window significantly diverge from expectation.

IHRs of a reference alignment exhibit various “signatures” that may be detected and/or identified after an initial alignment. For example, features such as abnormal read pairing, mismatches, and soft clipping may be detected and analyzed statistically. Regions in which these features exceed the expected background level may be identified as IHRs (lacking sufficient homology to the sample being sequenced) and thus being at risk for spurious variant calls.

IHRs may have a degree of homology relative to a particular genomic sequence that is in between very high homology and very low homology. Regions of reference sequence with very high homology to a set of sample sequence reads will allow alignment of said sequence reads and will subsequently produce very few variant calls. In contrast, regions of reference sequence with very low homology to a set of sample sequence reads will not allow alignment of said sequence reads to the reference sequence. However, IHRs can be problematic in the context of sequence alignment because sequence reads may be similar enough to the reference in these regions so as to permit alignment, albeit incorrect alignment that results in spurious variant calls that do not reflect the true sequence of the sample. IHRs can be patient specific, sequencing run-specific, and/or sample-specific.

In some embodiments, the present invention estimates the likelihood of an observed alignment pattern of a sequence read given it is aligned against a reference sequence of high homology. Sequence alignment programs (“aligners”) typically provide an alignment pattern result in the form of a CIGAR string which describes the number of matches, mismatches, insertions, and deletions that may be present in the alignment at a given region. Additional information which can be generated includes the length of soft-clipping (zero being no soft-clipping), and whether the sequence read has an abnormally paired mate. Under a null model of the reference sequence being highly homologous, each type of alignment anomaly, i.e., non-match pattern (mismatches, insertions, deletions, soft-clipping and abnormal pairing) should be independent and follow a Poisson distribution with some background rate. The unexpectedness of each of these features can be first evaluated independently and then statistically combined to estimate an overall per-read unexpectedness value, for example using Fisher's p-value combination method.

The exact background rate at which alignment anomalies occur can be sample, technology and/or sequencing run-specific. For example, two different sequencing runs may generate different distributions of quality scores, quality score-specific mismatch rates, or soft-clipping lengths. Thus, in some embodiments, accurate background estimates are obtained for each alignment anomaly type. Background rates for each anomaly type may be calculated, for example, based on the initial alignment data.

In some embodiments, unexpectedness (e.g., p-value) can be measured for each sequence read for each alignment anomaly type (e.g., soft-clip, mismatch, insertion, deletion, abnormal pairing). For each sequence read, the overall unexpectedness can be calculated based on the estimated sample and sequencing run-specific background values. In other words, the extent of abnormality of a particular combination of features exhibited by a sequence read can be determined for each sequence read. In some embodiments, the individual p-values are combined using Fisher's method to yield the final p-value that indicates how unlikely it is to observe the exact alignment pattern for each sequence read, assuming that each sequence read was aligned against a highly homologous reference sequence and all of its errors were generated independently from the background distribution. The present invention is preferable over other available alternatives because the present invention can determine if a sequence read is abnormal given the background distribution of all reads, and does not requires additional training of an algorithm or parameter estimation.

In some embodiments, as each sequence read in the alignment has an associated combined p-value, the combined p-values can be averaged using a smoothing function across windows of the reference alignment. Regions of the reference genome that are averaged in excess of a certain value may be identified as IHRs. In some embodiments, this may be further visualized as an additional “track” in the alignment.

Advantageously, the present invention can help a user to detect IHRs in a reference sequence with low false-negative results such as IHRs that would otherwise not have been detected using existing approaches, such as GATK.

“Genomic sequence data” herein means digital nucleic sequence information that provides a user with the genomic DNA sequence information of a subject. The genomic sequence data may be information that is stored in an electronic format, for example in a subject's medical records, or obtained from a gene sequencing apparatus, for example, a whole genome DNA sequencer.

“Reference sequence” herein means digital nucleic sequence information, either publicly available or proprietary sequence information, that provides a user with a representative example of a species' set of genes. In some embodiments, reference sequence may comprise sequences that are representative of the human genome. In one exemplary embodiment, the human genome reference sequence may be GRCh38. Reference sequence may represent nucleic sequences for an entire genome, or a portion of a genome.

“Local assembly” herein means an assembly of sequence reads into longer contigs, which may be limited to certain regions of the genome, for example, regions where the reference genome has insufficient homology with respect to the subject sample sequence. A local assembly may be de novo, i.e., the sequence reads are assembled without the aid of a reference sequence. De novo local assembly may be achieved by use of software or algorithms that assemble sequence reads into longer contigs or scaffolds by aligning overlapping ends of the sequence reads. Alternately, a local assembly may also incorporate the reference sequence.

“Remedial action” herein means actions or steps that are taken by a user of any of the embodiments of the invention in response to identification of IHRs of the reference sequence with respect to a genomic sample of a subject. Remedial action may be taken to increase accuracy, speed, and/or efficiency of genome sequencing and/or otherwise improve genome sequencing. Remedial actions can include, for example, masking the sequences of IHRs prior to making variant calls, performing local assembly of sequence reads aligned to locations within or nearby the IHR, and/or flagging the IHRs for needed improvement of reference sequences in future use.

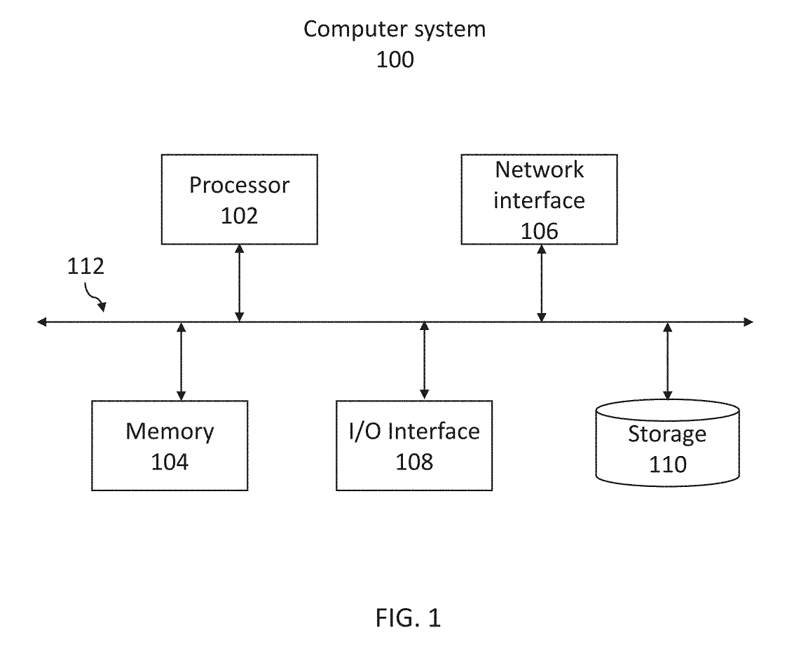

A person skilled in the art will recognize a variety of different computer-based technologies that can be used to carry out disclosures contained herein. For example, the devices, systems and methods disclosed herein can be implemented using one or more computer systems, such as the exemplary embodiment of a computer system 100 shown in FIG. 1.

As shown in FIG. 1, the computer system 100 can include one or more processors 102 which can control the operation of the computer system 100. The processor(s) 102 can include any type of microprocessor or central processing unit (CPU), including programmable general-purpose or special-purpose microprocessors and/or any one of a variety of proprietary or commercially available single or multi-processor systems. The computer system 100 can also include one or more memories 104, which can provide temporary storage for code to be executed by the processor(s) 102 or for data acquired from one or more users, storage devices, and/or databases. The memory 104 can include read-only memory (ROM), flash memory, one or more varieties of random access memory (RAM) (e.g., static RAM (SRAM), dynamic RAM (DRAM), or synchronous DRAM (SDRAM)), and/or a combination of memory technologies.

The various elements of the computer system 100 can be coupled to a bus system 112. The illustrated bus system 112 is an abstraction that represents any one or more separate physical busses, communication lines/interfaces, and/or multi-drop or point-to-point connections, connected by appropriate bridges, adapters, and/or controllers. The computer system 100 can also include one or more network interface(s) 106, one or more input/output (TO) interface(s) 108, and one or more storage device(s) 110.

The network interface(s) 106 can enable the computer system 100 to communicate with remote devices (e.g., other computer systems) over a network, and can be, for example, remote desktop connection interfaces, Ethernet adapters, and/or other local area network (LAN) adapters. The IO interface(s) 108 can include one or more interface components to connect the computer system 100 with other electronic equipment. For example, the IO interface(s) 108 can include high speed data ports, such as USB ports, 1394 ports, etc. Additionally, the computer system 100 can be accessible to a human user, and thus the IO interface(s) 108 can include displays, speakers, keyboards, pointing devices, and/or various other video, audio, or alphanumeric interfaces. The storage device(s) 110 can include any conventional medium for storing data in a non-volatile and/or non-transient manner. The storage device(s) 110 can thus hold data and/or instructions in a persistent state (i.e., the value is retained despite interruption of power to the computer system 100). The storage device(s) 110 can include one or more hard disk drives, flash drives, USB drives, optical drives, various media cards, and/or any combination thereof and can be directly connected to the computer system 100 or remotely connected thereto, such as over a network. The elements illustrated in FIG. 1 can be some or all of the elements of a single physical machine. In addition, not all of the illustrated elements need to be located on or in the same physical or logical machine. Rather, the illustrated elements can be distributed in nature, e.g., using a server farm or cloud-based technology. Exemplary computer systems include conventional desktop computers, workstations, minicomputers, laptop computers, tablet computers, PDAs, mobile phones, smartphones, and the like.

Although an exemplary computer system is depicted and described herein, it will be appreciated that this is for the sake of generality and convenience. In other embodiments, the computer system may differ in architecture and operation from that shown and described herein. For example, the computer system may be directly connected to a sequencing system or may be a part of a sequencer system.

In various aspects, the invention provides a computer-implemented method for identifying an insufficient homology region within a reference sequence with respect to a genomic sample of a subject, the method being based on determining the extent of abnormality of a particular combination of features exhibited by an aligned sequence read, the method comprising: (a) obtaining an initial alignment based on a reference sequence, the initial alignment comprising a plurality of sequence reads derived from the genomic sample of the subject and the reference sequence; (b) determining an expected background level for each of two or more types of alignment abnormalities in the initial alignment, the background levels being specific to the genomic sample; (c) determining an unexpectedness of observed alignment abnormalities for each sequence read by calculating a score based on the two or more types of observed alignment abnormalities in each sequence read; (d) identifying the insufficient homology region within the reference sequence, wherein the insufficient homology region is associated with sequence reads having a lower combined score than a threshold score; and (e) tagging the identified insufficient homology region for a remedial action.

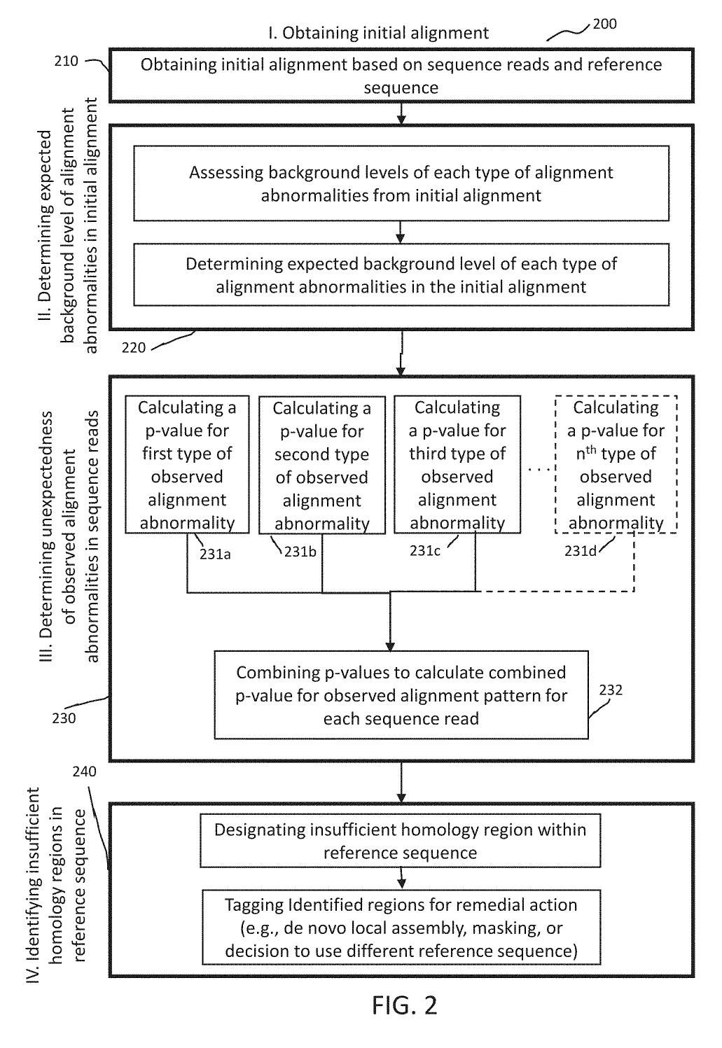

FIG. 2 illustrates a flowchart 200 for identifying IHRs within a reference sequence with respect to a genomic sample of a subject. As described above, a system 100 can be used to execute the method illustrated by the flowchart 200 of FIG. 2 to identify IHRs within a reference sequence with respect to a genomic sample of a subject.

As shown in FIG. 2, at step 210, an initial alignment can be obtained. The initial alignment may comprise a reference sequence and a plurality of sequence reads that are derived from a genetic sample of a subject. In some embodiments, the initial alignment may be obtained from one of the many publicly or commercially available aligners, or by a proprietary aligner. In some embodiments, the initial alignment may be generated by a sequencing platform. In other cases, the initial alignment may be part of a patient's medical records.

As shown in FIG. 2, at step 220, the expected background level of alignment abnormalities can be determined based on the information provided by the initial alignment of step 210. In some embodiments, the alignment abnormalities can be two or more different types of alignment abnormalities chosen from, but not limited to, the following: (a) abnormal read pairing type alignment abnormality; (b) soft clipping type alignment abnormality; (c) mismatch type alignment abnormality; (d) insertion type alignment abnormality; and (e) deletion type alignment abnormality. The expected background levels of each of the different types of alignment abnormalities can be determined separately.

The process of determining expected background levels of the different types of alignment abnormalities can depend on the type of alignment abnormalities. For example, if the alignment abnormality is a mismatch type abnormality, then the expected background level of the alignment abnormality may be determined based on (1) base position of the abnormality with respect to the sequence read; and (2) base quality of the base position of the abnormality. For another example, if the alignment abnormality is an insertion type abnormality or a deletion type abnormality, then the expected background levels of the alignment abnormality is determined based on a base position of the abnormality with respect to the sequence read. For yet another example, if the alignment abnormality is an abnormal read pairing type abnormality, then the step of determining the expected background levels comprise assessing the expected background level of the alignment abnormality by dividing a total count of the abnormality by a total number of the sequence reads. For still further yet another example, if the alignment abnormality is a soft-clipping type of abnormality, then the expected background levels of the alignment abnormality may be determined based on (a) length of a soft clip of a read; and (b) determination of whether the abnormality occurs at the 3′ end of the read or the 5′ end of the read.

As shown in FIG. 2, at step 230, an unexpectedness of observed alignment abnormalities can be determined for each of the sequence reads. In some embodiments, an unexpectedness of observed alignment abnormalities may be calculated by calculating a p-value for the different types of observed alignment abnormalities (231a, 231b, 231c, and 231d) and combining the resultantp-values to calculate a combined p-value for the unexpectedness of the observed pattern of alignment abnormalities 232.

The process of calculating individual p-values of the different types of observed alignment abnormalities can depend on the type of alignment abnormalities. For example, the p-value for observing a mismatch type may be calculated as follows: p=Pr(X≥o), wherein X˜Poisson(Σ_{i∈A}λᵢ), A={i: −10 log₁₀(λᵢ)≥20}, and o=Σ_{i∈A}I(mismatch at position i), λᵢ is the base quality at position i and I(⋅) is the indicator function. (The set A in this example comprises all positions having a base quality more than 20, though one may also vary this threshold.) As another example, the p-value for observing a soft clip type alignment abnormality may be calculated as follows: p=Σ_{i∈A}rᵢ, wherein A={i:rᵢ≤rₓ}, wherein p is calculated separately for the first and second reads and 5′ and 3′ ends of the sequence reads, and rᵢ is the rate at which the order and end of the reads are clipped for i bases. For yet another example, the p-value for observing an abnormal pairing type alignment abnormality may be calculated as follows: p=1 if the sequence read is free of an abnormality, and p=r_{abnormal read pairing} if the sequence read contains the abnormality, where r_{abnormal read pairing} is the overall rate of a mapped read pair being abnormally paired. For yet another example, the p-value for observing an indel type alignment abnormality may be calculated as follows: p=Pr(X≥o), wherein o is the number of indels observed in a read, X˜Poisson(Σᵢλᵢ) and λᵢ are the respective background likelihoods for having an indel aligned (i.e., non-soft-clipped) at position i, and wherein background rates for indels are computed separately for each of the two strands of the read.

As shown in FIG. 2, at step 232, the plurality of individual p-values for each of the types of observed alignment abnormalities can be combined to calculate a single combined p-value to represent the unexpectedness of observed alignment abnormalities in each of the sequence reads. In some embodiments, the plurality of individual p-values are combined to generate a single combined p-value using Fisher's method. In other embodiments, some of the individual p-values are combined first, and the resultant combined p-value is further combined with other individual p-values, so that the process of combining a plurality of individual p-values comprises a plurality of steps such that at each step, some individual p-values are combined first and the resultant intermediate combined p-values are combined with some or all of remaining individual p-values. In one preferred embodiment, the individual p-values are combined in the following steps: (a) combine mismatch and indel p-values; combine soft clipping p-value; (b) combine the resultant combined p-value with soft-clipping p-value; and (c) combine the resultant combined p-value with abnormal pairing p-value. In a preferred embodiment, the step of calculating the combined p-values can further comprise smoothing the combined p-value for the observed alignment abnormalities of each sequence window.

As shown in FIG. 2, at step 240, IHRs in a reference sequence may be designated based on the combined p-values for sequence reads 241. IHRs may be those regions that are associated with sequence reads having a lower combined score than a threshold score. In a preferred embodiment, IHRs are identified using per-read abnormality p-values as described above. In some embodiments, the identified IHRs can be tagged for a remedial action 242. In a preferred embodiment, the remedial action can be (a) a de novo local assembly of the identified insufficient homology region; (b) a masking of sequences of the IHRs for sequence analysis; or (c) a determination by a user on the appropriateness of the reference sequence for use with respect to the genomic sample of the subject.

The following examples are illustrative and not restrictive. Many variations of the technology will become apparent to those of skill in the art upon review of this disclosure. The scope of the technology should, therefore, be determined not with reference to the examples, but instead should be determined with reference to the appended claims along with their full scope of equivalents.

### EXAMPLES

### Example 1

A “per-read” approach of determining the likelihood of observing different types of alignment abnormality features of sequence reads in an initial alignment.

Calculated likelihood of observing different types of alignment abnormalities is shown in an exemplary hypothetical initial alignment between a reference genome and sequence reads of a subject.

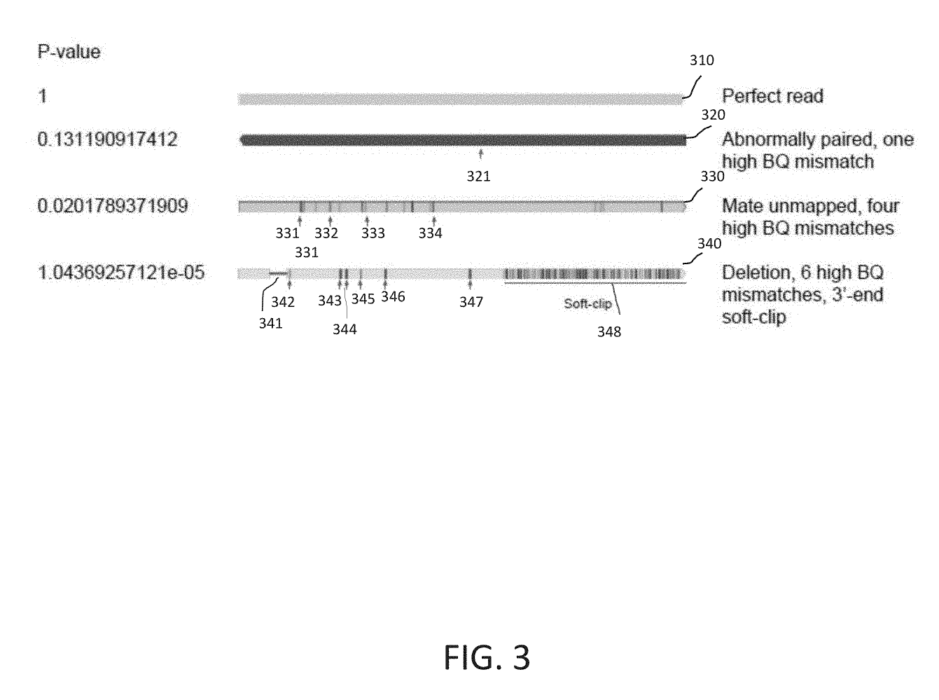

As shown in FIG. 3, various alignment abnormalities were identified in sequence reads from an initial alignment. In the first sequence read 310 shown, no alignment abnormalities were identified (i.e., there was a 100% match between the DNA sequence of the sequence read 310 and corresponding sequences in the reference sequence). In the second sequence read 320 shown, one high base quality mismatch 321 was observed. The probability of observing such pattern of alignment abnormalities (i.e., finding one mismatch), based on the background mismatch rate that was determined from the initial alignment, was calculated to be p=0.131190917412. In the third sequence read 330 shown, four high base quality mismatches 331, 332, 333, 334 were observed. The probability of observing such pattern of alignment abnormalities (i.e., observing four mismatches), based on the background mismatch rate that was determined from the initial alignment, was calculated to be p=0.0201789371909. In the fourth sequence read 340 shown, one deletion 341, six high base quality mismatches 342-347, and a 3′ end soft-clip 348 were observed. The probability of observing such pattern of alignment abnormalities (e.g., observing one deletion, six mismatches, and one 3′ end soft-clip), based on the background rate for each of the alignment abnormality types observed in the initial alignment, was calculated to be p=1.04369257121×10⁻⁵.

### Example 2

A comparison between IHRs identified using GATK HaplotypeCaller (i.e., Active Regions) and IHRs identified using an algorithm according to the present invention are shown for a reference sequence for a specific sample.

An initial alignment of sample genetic sequences against a reference genome was used to compare IHRs of the reference genome identified using GATK HaplotypeCaller (i.e., Active Regions) and IHRs of the reference genome identified using an algorithm according to the present invention.

Briefly, the genetic sample sequences were derived from a PCR-free sequencing run of sample NA12878, produced as part of the 1000 Genomes project and aligned to the GRCh38 reference genome using the software Burrows-Wheeler Aligner BWA MEM. The IHRs of the reference genome were identified using an algorithm as described elsewhere in the patent application and compared against IHRs (Active Regions) that were identified using GATK HaplotypeCaller. IHRs identified using GATK HaplotypeCaller are shown in red, whereas IHRs identified using an algorithm according to the present invention are shown by higher “low homology score,” shown in blue bars.

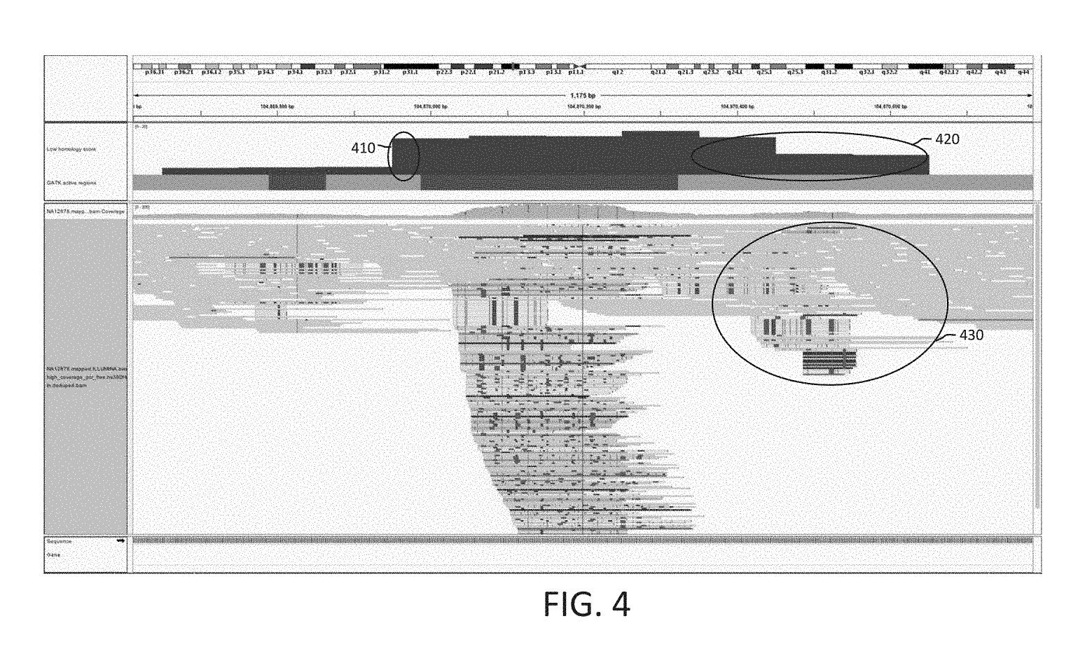

As shown in FIG. 4, an algorithm according to the present invention identified IHRs 410, 420 of a reference sequence that the currently available software GATK HaplotypeCaller failed to identify. Without wishing to be bound by any particular theory, the algorithm according to the present invention can identify previously undetected IHRs because the present invention provides for an algorithm that recognizes IHRs when the frequency of variants in a given region (e.g., sequence read) is higher than the background, while GATK HaplotypeCaller recognizes IHRs when there is a variant. In other words, GATK HaplotypeCaller tries to detect mismatch (genetic+sequencing error) rates that exceed that which is expected from sequencing errors, while the present invention is designed to allow for accurate detection of regions with a higher genetic variant rate than the expected background variant rate.

### Example 3

Identification of IHRs containing different types of alignment abnormalities, including abnormal pairings.

An initial alignment of sample genetic sequences against a reference genome was obtained as described above in Example 2.

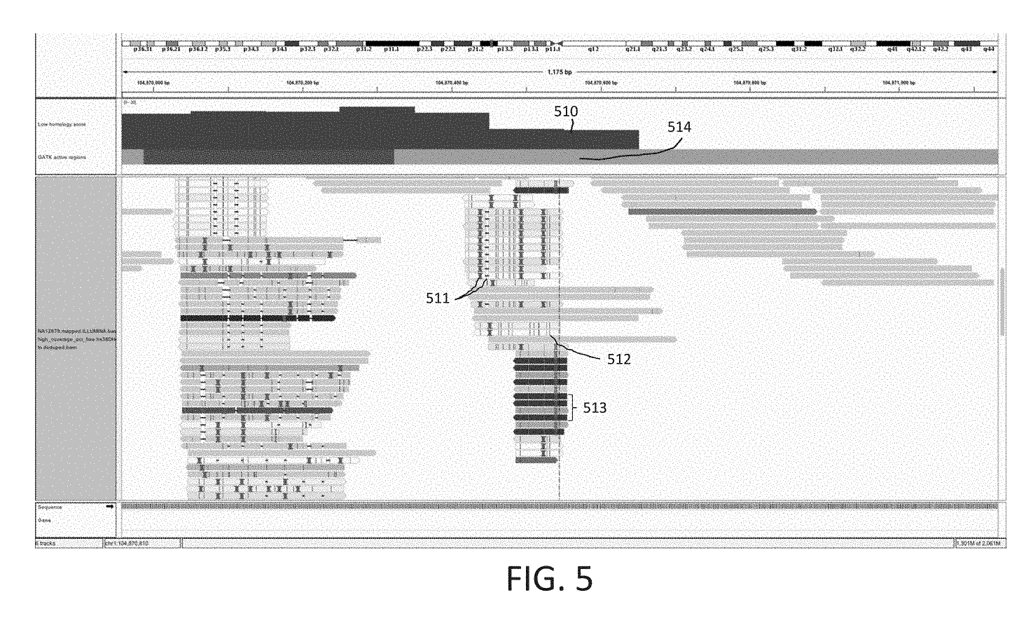

As shown in FIG. 5, a higher-resolution alignment depiction of a portion of the initial alignment data 430 shown in FIG. 4 shows that IHRs 510 identified using an algorithm according to the present invention correspond to sequence reads containing various types of alignment abnormalities such as indels 511, mismatches 512, abnormally paired reads 513 and soft-clipped from both the left and the right end (not shown). GATK HaplotypeCaller failed to identify these IHRs 515.

### Example 4

A reference sequence may have IHRs identified by GATK HaplotypeCaller but still be suitable for use in reference-assisted assembly.

An initial alignment of sample genetic sequences against a reference genome was obtained as described above in Example 2.

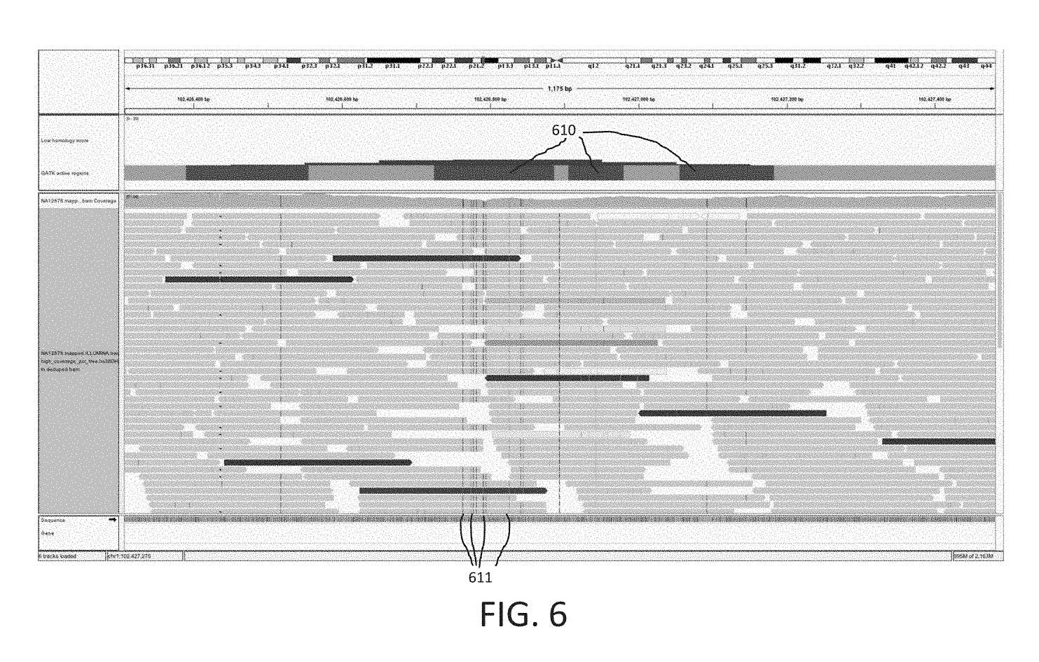

As shown in FIG. 6, regions of reference sequence were identified as being IHRs 610 by GATK HaplotypeCaller due to the presence of multiple SNPs 611. However, according to an algorithm of the present invention, the homology score is low in these regions (i.e., the regions are not considered IHRs) because only SNPs are present without indels, soft-clips or abnormal read pairing. Accordingly, the present invention can accurately identify exemplary regions of the reference sequence that are not IHRs, whereas other currently available alternatives, such as GATK HaplotypeCaller, may identify the regions as being IHRs. Without wishing to be bound by any particular theory, this discrepancy between the present invention and HaplotypeCaller can be attributed to the present invention being able to calculate a homology score based on the two or more types of observed alignment abnormalities in each sequence read by, for example, calculating an individual p-value for each of two or more types of observed alignment abnormalities in each sequence read; and combining a plurality of individual p-values to generate a single combined p-value for the observed alignment abnormalities for each sequence read. Additionally, without wishing to be bound by any particular theory, this discrepancy between the present invention and HaplotypeCaller can also be attributed to the present invention being able to detect regions with a higher genetic variant rate that takes into account sequencing error, for example, by calculating the background rate of alignment abnormalities from an initial alignment that is specific for each sequencing sample.

### Example 5

A reference sequence may have regions that are identified as IHRs by GATK HaplotypeCaller that are suitable for use in reference-assisted assembly.

An initial alignment of sample genetic sequences against a reference genome was obtained as described above in Example 2.

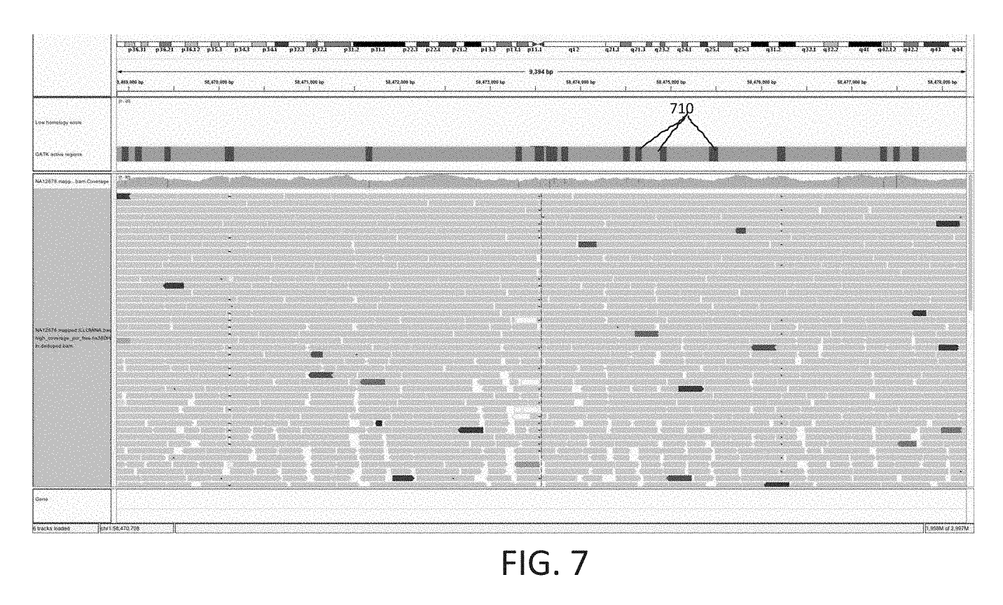

As shown in FIG. 7, GATK HaplotypeCaller identified IHRs 710 in the reference sequence where an algorithm according to the present invention did not identify IHRs. These regions show very a very homology score as calculated by an algorithm according to the present invention (i.e., sequence reads corresponding to these regions do not have an unexpectedly high rate of alignment abnormalities compared to the background rate of alignment abnormalities). Therefore, these regions of the reference sequence with a very low homology score can be used as a part of the reference sequence in a reference-assisted assembly because they do not contain frequencies of variants that are higher than the background. Therefore, the algorithm according to the present invention can identify regions of a reference sequence that can be used for reference-assisted assembly, even when those regions are identified as IHRs by other methods such as GATK HaplotypeCaller, because the present invention can detect regions with a higher genetic variant rate than that expected from background genetic variant rate whereas other presently available alternatives such as GATK detects per-base mismatch rate that can include sequencing errors.

1. A computer-implemented method for identifying an insufficient homology region within a reference sequence with respect to a genomic sample of a subject, the method being based on determining the extent of abnormality of a particular combination of features exhibited by an aligned sequence read, the method comprising:
(a) obtaining an initial alignment based on a reference sequence, the initial alignment comprising a plurality of sequence reads derived from the genomic sample of the subject and the reference sequence;
(b) determining an expected background level for each of two or more types of alignment abnormalities in the initial alignment, the background levels being specific to the genomic sample;
(c) determining an unexpectedness of observed alignment abnormalities for each sequence read by calculating a score based on the two or more types of observed alignment abnormalities in each sequence read;
(d) identifying the insufficient homology region within the reference sequence, wherein the insufficient homology region is associated with sequence reads having a lower combined score than a threshold score; and
(e) tagging the identified insufficient homology region for a remedial action.

2. The method of claim 1, wherein the initial alignment comprises a reference-assisted assembly of the sequence reads to the reference sequence.

3. The method of claim 1, wherein the types of observed alignment abnormalities comprise at least two of: (a) abnormal read pairing type alignment abnormality; (b) soft clipping type alignment abnormality; (c) mismatch type alignment abnormality; (d) insertion type alignment abnormality; and (e) deletion type alignment abnormality.

4. The method of claim 3, wherein, if the alignment abnormality is a mismatch type abnormality, then the step of determining the expected background levels comprise assessing the expected background level of the alignment abnormality based on the following covariates:
(a) base position of the abnormality with respect to the sequence read; and
(b) base quality of the base position of the abnormality.

5. The method of claim 3, wherein, if the alignment abnormality is an insertion type abnormality or a deletion type abnormality, then the step of determining the expected background levels comprise assessing the expected background level of an alignment abnormality based on a base position of the abnormality with respect to the sequence read.

6. The method of claim 3, wherein, if the alignment abnormality is an abnormal read pairing type abnormality, then the step of determining the expected background levels comprise assessing the expected background level of the alignment abnormality by dividing a total count of the abnormality by a total number of the sequence reads.

7. The method of claim 3, wherein, if the alignment abnormality is a soft-clipping type of abnormality, then the step of determining the expected background levels comprises assessing the expected background level of the alignment abnormality based on the following covariates:
(a) length of a soft-clip of a read; and
(b) determination of whether the abnormality occurs at the 3′ end of the read or the 5′ end of the read.

8. The method of claim 1, wherein calculating a score based on the two or more types of observed alignment abnormalities in each sequence read comprises:
(a) calculating an individual p-value for each of two or more types of observed alignment abnormalities in each sequence read; and
(b) combining a plurality of individual p-values to generate a single combined p-value for the observed alignment abnormalities for each sequence read;
wherein the insufficient homology region has a lower combined p-value than a threshold p-value.

9. The method of claim 8, wherein the p-value of each type of observed alignment abnormality in each sequence read is calculated by performing one of:
(a) p=Pr(X≥o), wherein X˜Poisson(Σ_{i∈A}λᵢ), A={i: −10 log₁₀(λᵢ)≥20}, and o=Σ_{i∈A}I(mismatch at position i), λᵢ is the base quality at position i and I(⋅) is the indicator function;
(b) p=Σ_{i∈A}rᵢ, wherein A={i:rᵢ≤rₓ}, wherein p is calculated separately for the first and second reads and 5′ and 3′ ends of the sequence reads, and rᵢ is the rate at which the order and end of the reads are clipped for i bases;
(c) p=1 if the sequence read is free of an abnormality, and p=r_{abnormal read pairing} if the sequence read contains the abnormality, where r_{abnormal read pairing} is the overall rate of a mapped read pair being abnormally paired; and
(d) p=Pr(X≥o), wherein o is the number of indels observed in a read, X˜Poisson(Σᵢλᵢ) and λᵢ are the respective background likelihoods for having an indel aligned at position i, and wherein background rates for indels are computed separately for each of the two strands of the read.

10. The method of claim 8, wherein the step (b) is performed using Fisher's method.

11. The method of claim 10, wherein the step of combining a plurality of individual p-values comprises a plurality of steps such that at each step, some individual p-values are combined first and the resultant intermediate combined p-values are combined with some or all of remaining individual p-values.

12. The method of claim 8, wherein the step of generating the combined p-value further comprises smoothing the combined p-value for the observed alignment abnormalities of each sequence window.

13. The method of claim 1, wherein the remedial action comprises: (a) a de novo local assembly of the identified insufficient homology region; (b) a masking of sequences of the insufficient homology region; or (c) a determination by a user on the appropriateness of the reference sequence for use with respect to the genomic sample of the subject.

14-15. (canceled)

16. A data processing system for designating an insufficient homology region of a reference sequence, the system comprising:
at least one memory operable to store bioinformatics data; and
a processor communicatively coupled to the at least one memory, the processor being programmed to:
(a) obtain an initial alignment based on a reference sequence, the initial alignment comprising a plurality of sequence reads derived from the genomic sample of the subject and the reference sequence;
(b) determine an expected background level for each of two or more types of alignment abnormalities in the initial alignment, the background levels being specific to the genomic sample;
(c) determine an unexpectedness of observed alignment abnormalities for each sequence read by calculating a score based on the two or more types of observed alignment abnormalities in each sequence read;
(d) identify the insufficient homology region within the reference sequence, wherein the insufficient homology region is associated with sequence reads having a lower combined score than a threshold score; and
(e) tag the identified insufficient homology region for a remedial action.

17. The data processing system of claim 16, wherein the initial alignment comprises a reference-assisted assembly of the sequence reads to the reference sequence.

18. The data processing system of claim 16, wherein the types of observed alignment abnormalities comprise at least two of: (a) abnormal read pairing type alignment abnormality; (b) soft clipping type alignment abnormality; (c) mismatch type alignment abnormality; (d) insertion type alignment abnormality; and (e) deletion type alignment abnormality.

19. The data processing system of claim 18, wherein, if the alignment abnormality is a mismatch type abnormality, then the step of determining the expected background levels comprise assessing the expected background level of the alignment abnormality based on the following covariates:
(a) base position of the abnormality with respect to the sequence read; and
(b) base quality of the base position of the abnormality.

20. The data processing system of claim 18, wherein, if the alignment abnormality is an insertion type abnormality or a deletion type abnormality, then the step of determining the expected background levels comprise assessing the expected background level of an alignment abnormality based on a base position of the abnormality with respect to the sequence read.

21. The data processing system of claim 18, wherein, if the alignment abnormality is an abnormal read pairing type abnormality, then the step of determining the expected background levels comprise assessing the expected background level of the alignment abnormality by dividing a total count of the abnormality by a total number of the sequence reads.

22. A non-transitory computer-readable medium comprising instructions which, when implemented by one or more computers, cause the one or more computers to perform:
(a) obtaining an initial alignment based on a reference sequence, the initial alignment comprising a plurality of sequence reads derived from the genomic sample of the subject and the reference sequence;
(b) determining an expected background level for each of two or more types of alignment abnormalities in the initial alignment, the background levels being specific to the genomic sample;
(c) determining an unexpectedness of observed alignment abnormalities for each sequence read by calculating a score based on the two or more types of observed alignment abnormalities in each sequence read;
(d) identifying the insufficient homology region within the reference sequence, wherein the insufficient homology region is associated with sequence reads having a lower combined score than a threshold score; and
(e) tagging the identified insufficient homology region for a remedial action.
